(19) United States
(12) Patent Application Publication
(10) Pub. No.: US 2026/0331263 A1
(43) Pub. Date: Oct. 22, 2026

(54) Adaptive Hybrid Filtering and Quantum-Inspired Search Framework with Virtual Spectral Decomposition for Medical Image Analysis and Clinical Trial Optimization

(71) Applicant: Shubham Chandra, Katy, TX (US)

(72) Inventor: Shubham Chandra, Katy, TX (US)

(21) Appl. No.: 19/640,868

(22) Filed: Apr. 7, 2026

Related U.S. Application Data
(63) Continuation-in-part of application No. 19/315,529, filed on Aug. 30, 2025, now abandoned.
Application No. 19/315,529 is a continuation-in-part of application No. 19/183,878, filed on Apr. 20, 2025.

(51) Int. Cl. G16H 30/40; G06N 10/60; G06T 7/00; G16H 10/20; G16H 20/00; G16H 50/20

(52) U.S. Cl. CPC G16H 30/40; G06T 7/0012; G16H 10/20; G16H 20/00; G16H 50/20; G06N 10/60; G06T 2207/20081; G06T 2207/30041

(57) ABSTRACT

An adaptive biological analysis system integrating Virtual Spectral Decomposition with dendritic binary gating for medical image analysis and therapeutic combination design. The system decomposes standard RGB camera images into virtual spectral channels using parametric sigmoid transfer functions, then applies dendritic morphological gating to extract twelve physically interpretable biomarker features including vessel density, coherence, oxygen saturation proxy, cup-to-disc ratio, peripapillary vessel density, and lens opacity index. An adaptive triage layer classifies normal samples without deep learning inference, reducing computational cost by twenty to sixty percent. A Kill Zone Framework encodes biological defense pathways as ternary signatures, computes weighted Hamming distances to drug masks, and identifies minimum therapeutic combinations achieving distance zero across all active defenses, validated in pancreatic cancer with companion drug identification for failed clinical trial rescue.

### CROSS-REFERENCE TO RELATED APPLICATIONS

This application is a Continuation-in-Part of U.S. patent application Ser. No. 19/315,529, filed Aug. 30, 2025, entitled “Adaptive Hybrid Filtering and Quantum-Inspired Search Framework with Meta-Learning for Improved Information Retrieval in Noisy Environments,” which is itself a Continuation-in-Part of U.S. patent application Ser. No. 19/183,878, filed Apr. 20, 2025, entitled “Hybrid Filtering and Quantum-Inspired Search Framework for Improved Information Retrieval in Noisy Environments,” both of which are hereby incorporated by reference in their entirety.

This application is related to the following co-pending applications, each of which is incorporated by reference in its entirety: U.S. patent application Ser. No. 19/309,918, filed Aug. 26, 2025, entitled “Portable Retinal AI System for Multi-Disease Prediction and Personalized Therapy,” which is a Continuation-in-Part of U.S. patent application Ser. No. 19/080,535, filed Mar. 14, 2025, entitled “Hybrid AI-Powered Digital Clone for Real-Time Health Monitoring and Legacy Preservation”; U.S. patent application Ser. No. 19/543,206, filed Feb. 18, 2026, entitled “System and Method for Multi-Spectral Retinal Fundus Imaging and Wavelength-Differential Virtual Spectral Decomposition for Interpretable Ocular Disease Classification and Biomarker Extraction”; and U.S. patent application Ser. No. 19/553,050, filed Feb. 27, 2026, entitled “Retinal Fundus Analysis Using Virtual Spectral Decomposition.”

### FIELD OF THE INVENTION

The present invention relates to adaptive information retrieval, medical image analysis, and computational therapeutic design. More specifically, the invention relates to systems and methods that integrate biologically-inspired dendritic filtering with quantum-inspired symbolic search and meta-learning optimization, extended through Virtual Spectral Decomposition (VSD) for extraction of physically interpretable biomarkers from standard camera images, and further extended through a Kill Zone Framework for computing minimum therapeutic combinations to overcome biological defense architectures in cancer, viral infections, bacterial infections, and parasitic infections.

### BACKGROUND OF THE INVENTION

### A. Search and Filtering in Noisy Environments

Search algorithms are essential components of numerous computational systems, from fraud detection engines to genomic screening tools. In complex or noisy environments, conventional search models are unable to distinguish between signal and noise, leading to false positives, inefficiencies, and degraded performance. Quantum algorithms, such as Grover's search (Grover, 1996), offer theoretical quadratic speedup but remain susceptible to input noise and practical implementation limitations. Symbolic matchers relying on Hamming distance assume exactitude or fixed-tolerance levels that are insufficient in real-world sensor or perceptual data. Additionally, traditional systems lack the ability to learn from experience and adapt to changing data distributions over time.

Prior solutions include Grover's algorithm for quantum search, Hamming distance classifiers for symbolic matching, and percentile-based threshold filters. These systems operate independently and fail to incorporate adaptive biological filtering principles that optimize for relevance before computation. While some prior systems incorporate static threshold gates, they do not evolve based on training data distribution, do not guarantee preservation of true positive targets, and do not improve their performance through accumulated experience. Existing machine learning approaches typically require complete retraining when data distributions shift, lacking the continuous adaptation capabilities needed for real-world deployments.

### B. Retinal Imaging and Artificial Intelligence for Disease Detection

Retinal disease represents one of the most significant sources of preventable blindness worldwide. Diabetic retinopathy affects approximately 415 million individuals globally. Age-related macular degeneration affects more than 196 million individuals. Glaucoma affects an estimated 76 million individuals and causes irreversible optic nerve damage. The global ophthalmologist shortage is acute, with approximately one ophthalmologist per 186,000 people globally, and substantially worse ratios in developing regions. Standard-of-care fundus photography requires dedicated instruments costing $15,000 to $150,000, skilled operators, and clinical infrastructure inaccessible to the majority of at-risk populations.

Existing AI systems for retinal disease detection have achieved significant clinical success. IDx-DR (Digital Diagnostics), the first FDA-cleared autonomous AI diagnostic system, achieves high sensitivity for diabetic retinopathy screening. Google DeepMind's retinal analysis system demonstrated that fundus photographs contain signals predictive of cardiovascular risk factors (Poplin et al., Nature Biomedical Engineering, 2018). EyeArt from Eyenuk provides cloud-based diabetic retinopathy screening. The RETFound foundation model (Zhou et al., Nature, 2023) enables label-efficient retinal analysis across multiple conditions. However, all existing systems share a fundamental explainability limitation: their classification decisions are generated through opaque learned feature representations that cannot be directly mapped to physiological tissue properties or disease mechanisms.

Gradient-weighted Class Activation Maps (Grad-CAM), Integrated Gradients, SHAP values, and other post-hoc attribution methods provide spatial heatmaps indicating which image regions influenced the classification, but these attributions are expressed in pixel space rather than physiological space. An ophthalmologist reviewing a Grad-CAM overlay cannot determine whether the highlighted region reflects hemoglobin deoxygenation, capillary non-perfusion, microaneurysm formation, or artifact. The attribution tells the clinician where the model looked but not what the model detected or what tissue property changed. This opacity represents a material regulatory limitation under FDA's Software as a Medical Device (SaMD) framework and the European Medical Device Regulation (EU-MDR), which increasingly require demonstration of decision transparency, reproducibility, and post-market auditability.

Recent work by Dumitrascu et al. (Mayo Clinic Proceedings: Digital Health, 2024) demonstrated that a BERT-style self-supervised convolutional neural network (ADRET) pretrained on UK Biobank retinal color photographs achieved 98.27% accuracy for Alzheimer's disease classification from fundus images alone. While this demonstrates the diagnostic potential of retinal imaging for neurological disease, the ADRET model operates as a black-box classifier without physically interpretable biomarkers, without spectral decomposition, without camera-agnostic normalization, and without computational triage optimization. The model's attention heatmaps highlight regions surrounding small vascular branches but cannot specify which tissue property (vessel density, oxygen saturation, nerve fiber thickness) drove the classification decision.

U.S. Patent Application Publication No. US20190110753A1 describes basic deep learning for ophthalmic diseases but is limited to static models without few-shot adaptation or bidirectional inference. Optina Diagnostics has filed PCT applications for retinal Alzheimer's detection using hyperspectral imaging hardware, but their approach requires specialized cameras costing $50,000 or more and is not portable or camera-agnostic. The Oxymap T1 provides retinal oximetry at the research level but is large, expensive, and requires specialized operators. No existing portable system can simultaneously perform cataract severity assessment and retinal disease detection from a single imaging session.

### C. Therapeutic Combination Design and Drug Resistance

Current oncology is mutation-centric: sequence the tumor, find the mutation, match to a drug. This paradigm treats each mutation as an independent event and each drug as a single-target intervention. The mutation-centric paradigm asks “What is broken in this cell?” rather than “What can this cell do to survive?” Targets identified by mutation analysis can be circumvented through bypass mutations, leading to acquired resistance in the majority of treated patients.

Approximately 90% of oncology drugs entering clinical trials fail, representing billions of dollars in sunk development costs. The kill zone principle, wherein all defense mechanisms must be simultaneously addressed, has been empirically validated in HIV treatment (HAART: three drugs from three classes), tuberculosis (RIPE: four-drug regimen), and malaria (artemisinin-based combination therapy). However, no prior computational framework exists that systematically encodes an organism's defense architecture, computes the distance between that architecture and each available therapeutic agent, and designs the minimum combination needed to overcome all active defenses simultaneously.

Multi-state encoding of biological systems has been explored in Boolean network models and Kauffman's random Boolean networks for gene regulatory dynamics, but these systems do not incorporate the concept of defense pathways with therapeutic drug masks, do not compute Hamming distances to specific drug targets, and do not identify minimum combinations achieving distance zero. The present invention addresses all of these limitations.

### SUMMARY OF THE INVENTION

The present invention provides an integrated adaptive biological analysis system comprising three interconnected innovation layers built upon the parent application's dendritic filtering, quantum-inspired search, and meta-learning architecture.

In a first aspect, the invention provides a Virtual Spectral Decomposition (VSD) module that decomposes standard RGB camera images into virtual spectral channels approximating specific wavelength responses using parametric sigmoid transfer functions. The VSD module operates on any imaging sensor without hardware modification, enabling extraction of physiologically meaningful biomarkers from commodity cameras.

In a second aspect, the invention provides a Dendritic Binary Gating module that applies biologically-inspired morphological operations and adaptive thresholding to VSD channels, producing binary activation maps and twelve quantitative biomarker features with direct physical interpretability. The dendritic gating operates as a triage layer, routing high-confidence normal samples around computationally expensive deep learning inference.

In a third aspect, the invention provides a Kill Zone Framework for computing minimum therapeutic combinations by encoding biological defense pathways as ternary signatures (Active/Primed/Locked), computing weighted Hamming distances to drug masks, and identifying the minimum combination achieving distance zero across all active defenses. The framework includes trajectory analysis, lock identification, defense build order prediction, and companion drug identification for rescue of failed clinical trials.

In a fourth aspect, the invention provides clinical trial optimization applications including multi-site data harmonization through device-specific VSD calibration, automated patient stratification based on biomarker profiles, longitudinal biomarker trajectory monitoring, and adaptive surrogate endpoint detection.

In a fifth aspect, the invention provides an explainable artificial intelligence framework wherein every classification decision is traceable to specific tissue properties (for medical imaging) or specific defense pathway states (for therapeutic design), providing the decision transparency required for regulatory approval and clinical adoption.

### BRIEF DESCRIPTION OF THE DRAWINGS

FIG. 1 is a block diagram illustrating the overall architecture of the adaptive hybrid search system, showing the input data module, the dendritic filtering layer, the symbolic quantum-inspired search module, the meta-learning module, and the fallback logic layer.

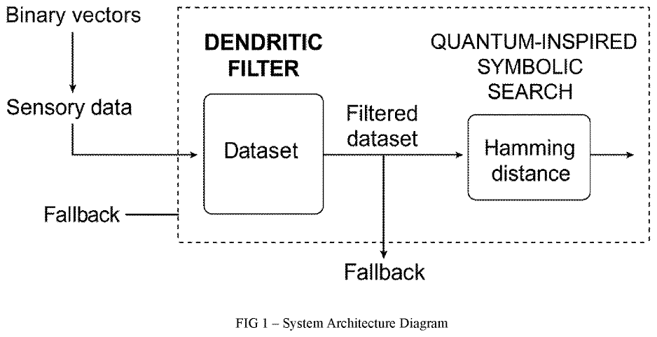

FIG. 2 is a performance analysis diagram showing precision-recall curves for both the unfiltered and dendritic-filtered search pipelines at multiple Hamming distance thresholds.

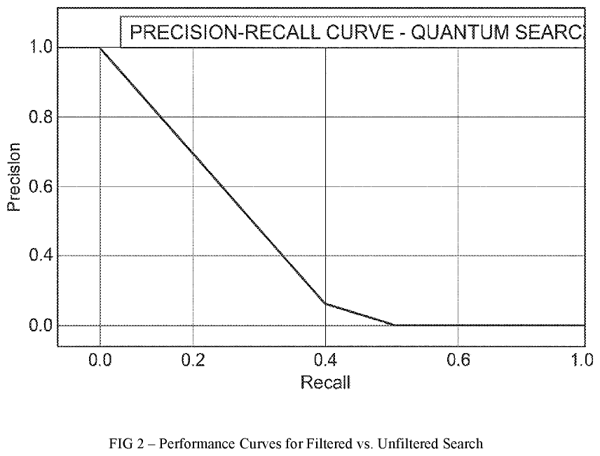

FIG. 3 is a pair of confusion matrices illustrating classification outcomes from the symbolic quantum search both with and without the dendritic filter, demonstrating increased precision and reduced false positives in the filtered configuration.

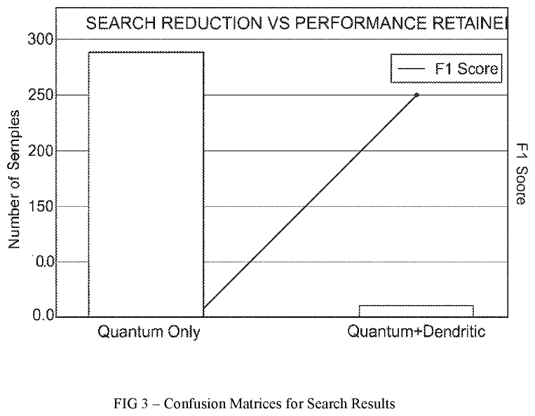

FIG. 4 is a colored layered schematic representing the hybrid architecture in an end-to-end format, depicting the flow from raw sensory input through adaptive dendritic gating into the symbolic quantum evaluator.

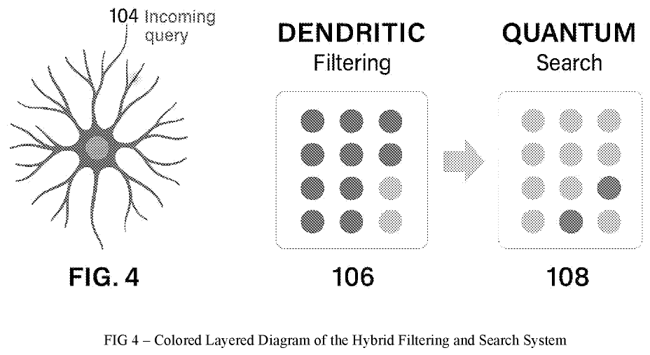

FIG. 5 illustrates the meta-learning module architecture showing the interconnection between the Historical Performance Database (502), Noise Characterization Engine (506), Strategy Selection Module (514), Performance Prediction Engine (520), and Continuous Learning Loop (526).

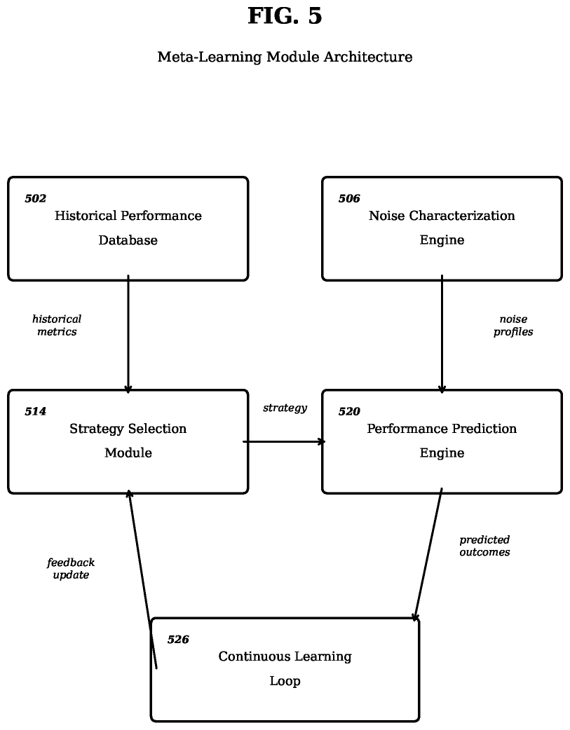

FIG. 6 depicts the temporal adaptation mechanism with three temporal models operating at different timescales: short-term (100-sample window), medium-term (1000-sample window), and long-term (10000-sample window) with weighted combining based on detected drift rates.

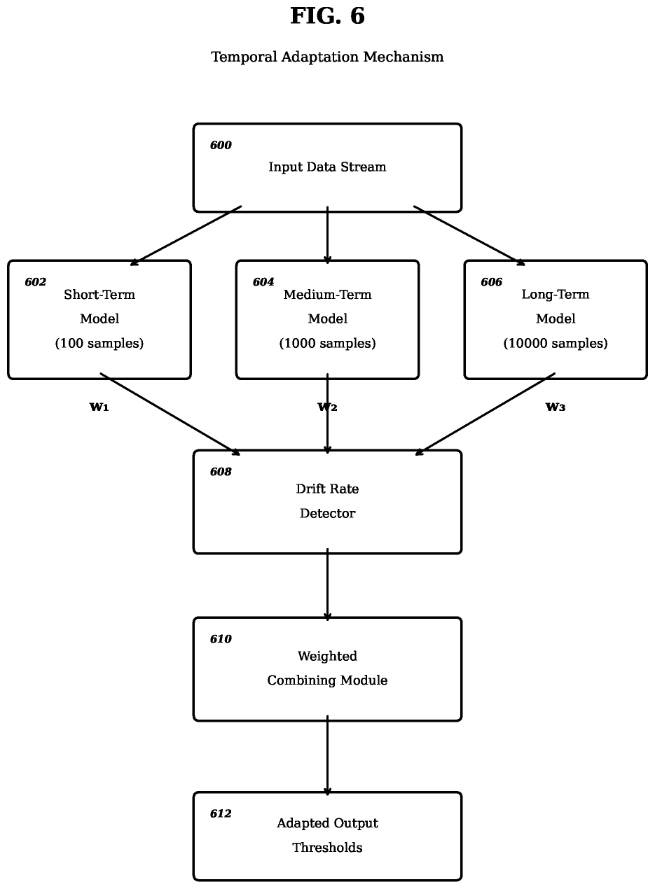

FIG. 7 shows the transfer learning architecture enabling rapid domain adaptation through source domain models, domain adaptation layers using Maximum Mean Discrepancy and gradient reversal, few-shot learning modules, and zero-shot generalization modules.

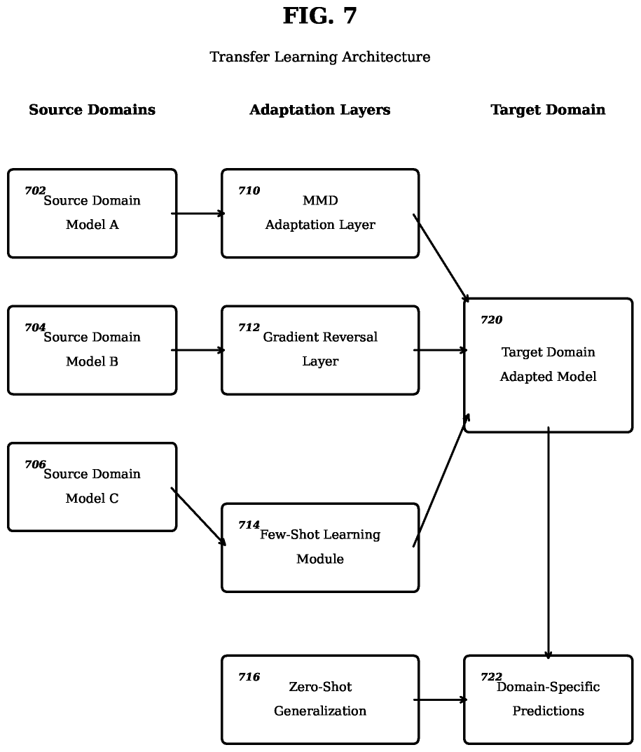

FIG. 8 illustrates performance improvement curves over time showing F1 score versus number of searches for baseline static thresholds, meta-learning without transfer, and meta-learning with transfer learning.

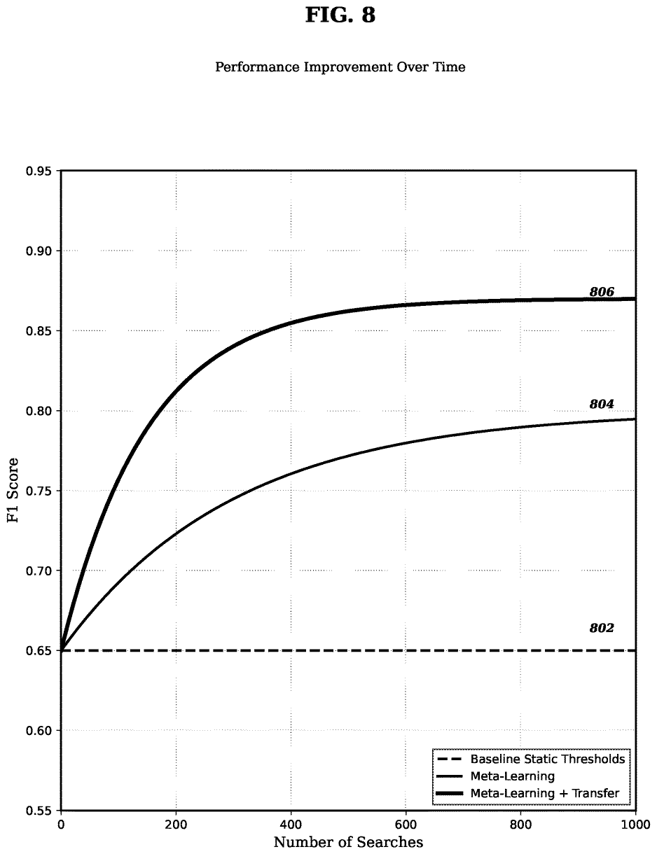

FIG. 9 provides a visual representation of the biomarker identification process in genomic applications showing four stages: input layer with genomic data points, dendritic filtering layer removing noise, symbolic quantum matcher identifying patterns, and output layer with stable biomarkers.

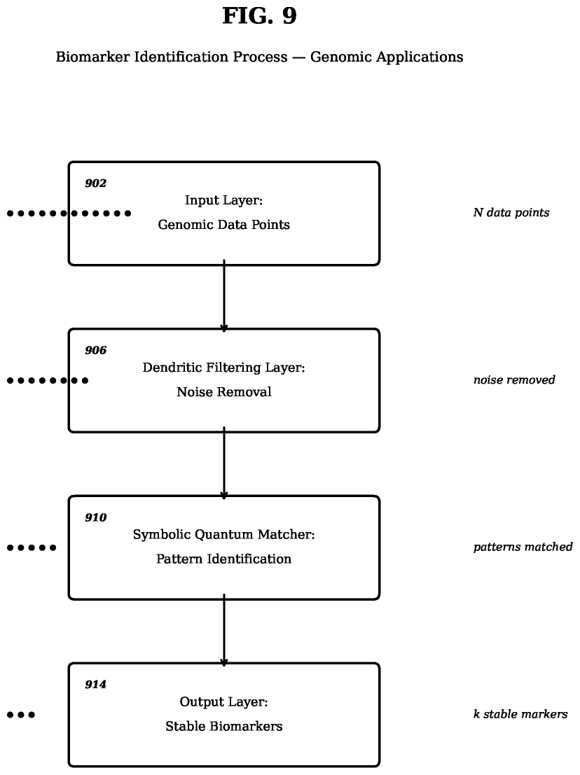

FIG. 10 illustrates the wearable sensor data processing pipeline showing input layer with multi-modal sensor data, dendritic filter with adaptive gates, symbolic matcher for real-time pattern detection, and output display with personalized health insights.

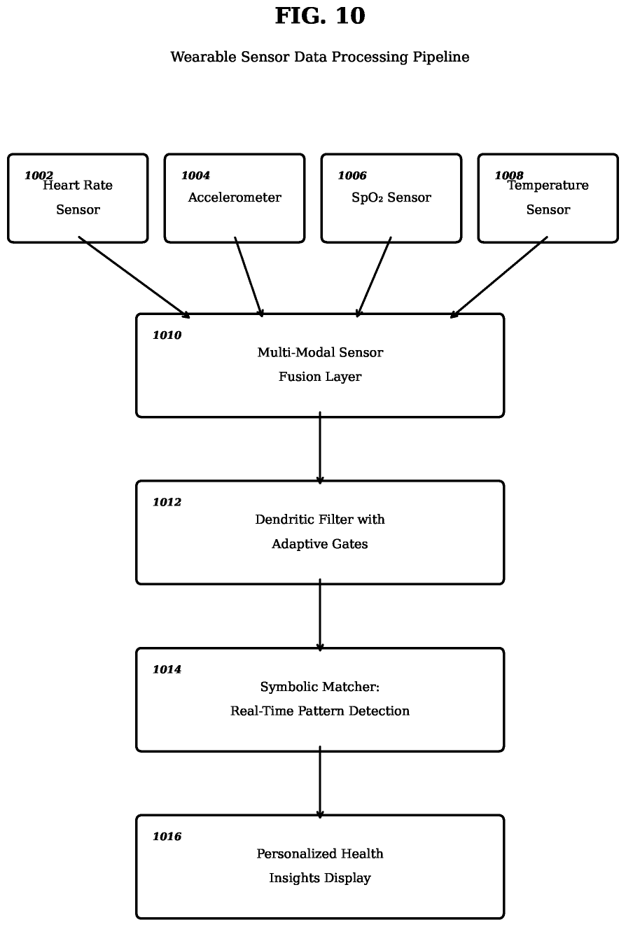

FIG. 11 depicts the canine health monitoring pipeline showing a canine subject with sensor collar, input module, dendritic filtering stage tuned to canine physiology, symbolic matching module, and primary and secondary output displays.

FIG. 12 is a block diagram illustrating the VSD-Dendritic integrated architecture for retinal image analysis, showing: RGB fundus image input (1200); VSD decomposition module (1202) producing six virtual spectral channels; Dendritic Binary Gating module (1204) producing binary firing maps; biomarker extraction module (1206) computing twelve features; triage decision point (1208); normal classification bypass (1210); CNN backbone (1214) with 2048-dimensional features; biomarker embedding network (1216) with 128-dimensional output; fusion classification head (1218); and disease-specific boosting module (1220).

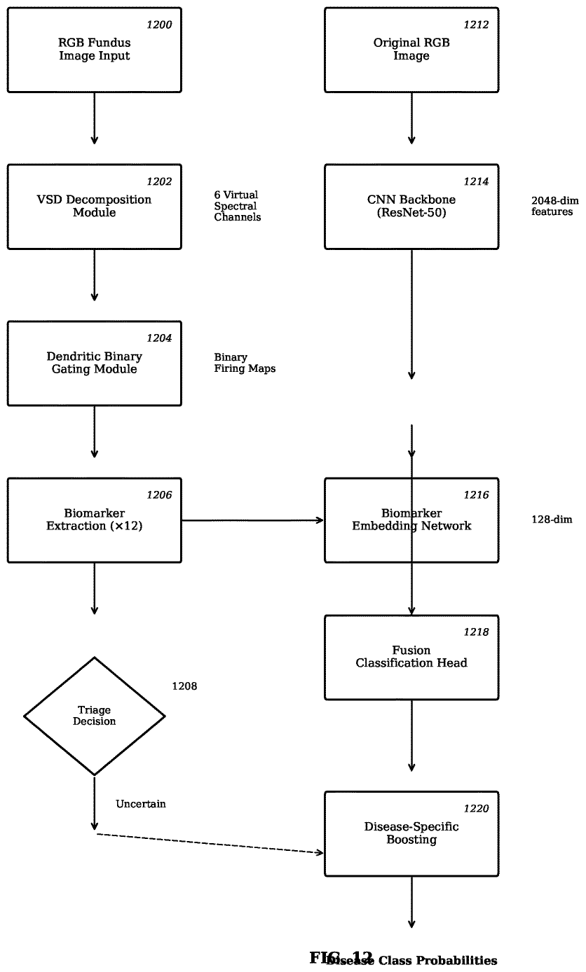

FIG. 13 is a flowchart illustrating clinical trial data harmonization, showing: fundus images from multiple camera types at multiple sites (1300-1304); per-device VSD parameter calibration (1306-1310); unified VSD channel generation (1312); dendritic binary gating producing device-invariant biomarkers (1314-1316); patient stratification (1318); longitudinal trajectory monitoring (1320); and adaptive endpoint detection (1322) with meta-learning feedback.

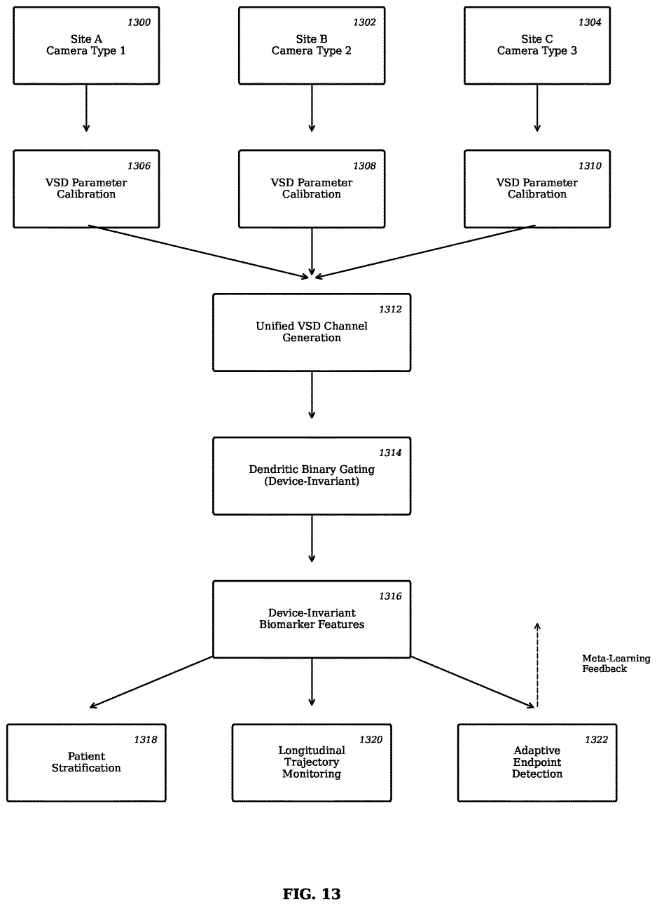

FIG. 14 is a block diagram of the Kill Zone Framework architecture, showing: molecular profiling input (1400); dendritic cascade module (1402) processing pathways in drug-specific discriminability order; ternary signature generation (1404); drug mask definitions (1406); weighted Hamming distance computation (1408); kill zone identification (1410); trajectory analysis (1412); companion drug identification for failed trial rescue (1414); and meta-learning feedback loop.

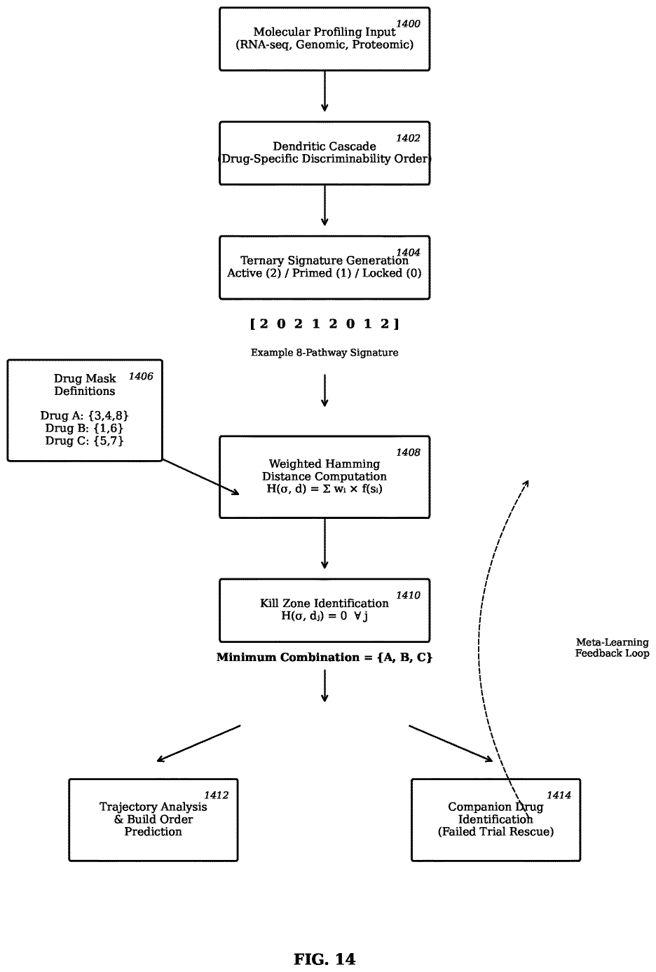

FIG. 15 is a defense build order diagram for pancreatic ductal adenocarcinoma showing the fraction of patients with each of eight defense pathways in the Active state at each total defense count from one through eight, demonstrating that apoptosis resistance activates earliest while drug metabolism and immune evasion activate latest.

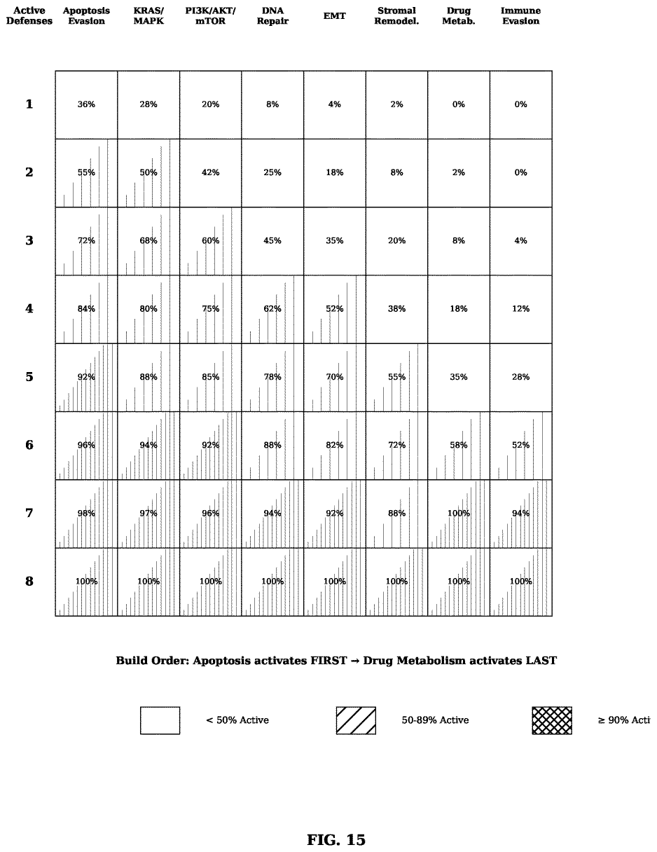

FIG. 16 is a lock combination synergy matrix showing hazard ratios for individual pathway locks on the diagonal and pairwise lock combinations off-diagonal, with hatching patterns indicating strength of synergistic protection.

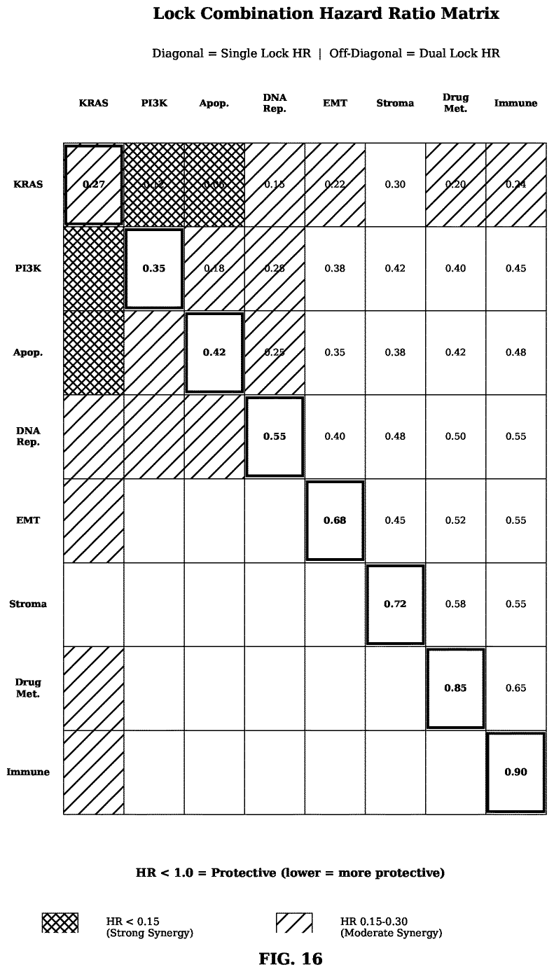

### DETAILED DESCRIPTION OF THE INVENTION

### Part I: Adaptive Hybrid Filtering and Quantum-Inspired Search Architecture

The invention builds upon the parent application's three-component architecture: (1) a dendritic filtering module that applies learned and continuously refined thresholds to sensory data, (2) a symbolic quantum search engine that identifies target sequences through bitstring comparison using Hamming distance, and (3) a meta-learning module that tracks performance history and optimizes system parameters over time. The complete parent disclosure is incorporated herein by reference.

Symbolic Quantum Search Module: The search operates on binary vectors (candidate inputs) and compares them against a target bitstring using Hamming distance as the primary metric. For a binary vector x=(x_1, x_2, . . . , x_n) and a target bitstring t=(t_1, t_2, . . . , t_n), the Hamming distance is defined as:

d_H(x, t)=SUM_i=1 . . . n[indicator(x_i!=t_i)] (Equation 1)
where the indicator function returns 1 if x_i differs from t_i and 0 otherwise. The algorithm employs an adaptive symbolic scoring mechanism that mimics amplitude amplification in Grover's algorithm:

s(x, t)=f(d_H(x, t))*w(historical_performance) (Equation 2)
where f is a monotonically decreasing function assigning higher scores to lower distances, and w is a weighting function derived from meta-learning based on historical success rates.

Dendritic Filtering Layer: The filtering layer processes continuous sensory features f_i and applies gating thresholds theta_i using conjunctive logic. The parent application discloses multiple threshold computation methods including percentile-based heuristics, performance-optimized thresholds, Bayesian optimization using Gaussian Process regression with Expected Improvement acquisition function, differential evolution strategies, ensemble methods combining multiple strategies with performance-weighted averaging, and meta-learned strategy selection.

Fallback Mechanism: To prevent over-filtering, the invention includes a fallback mechanism ensuring |D_f INTERSECT D_positive|>=1, meaning at least one positive target is always preserved in the filtered dataset through intelligent reinsertion when all positives are removed.

Meta-Learning Module: The meta-learning module maintains a Historical Performance Database (502) storing threshold configurations, performance metrics, and data characteristics across all search operations. A Noise Characterization Engine (506) analyzes incoming data through spectral analysis, statistical tests, and distribution fitting. A Strategy Selection Module (514) uses k-nearest neighbor search and Random Forest regression to predict optimal strategies. A Continuous Learning Loop (526) implements incremental updates every 100 searches with full model retraining when significant drift is detected.

Temporal Adaptation: Three temporal models operate simultaneously: a short-term model (602) with 100-sample window capturing immediate changes, a medium-term model (606) with 1000-sample window providing stability, and a long-term model (610) with 10000-sample window identifying seasonal patterns. A weighted combiner (614) merges predictions based on drift rates detected by a drift detector (618) implementing ADWIN, Page-Hinkley, and KSWIN algorithms.

Transfer Learning: The system implements domain adaptation through source domain models (702) with pre-trained thresholds and learned patterns, a domain adaptation layer (708) computing Maximum Mean Discrepancy (710) with gradient reversal layers (712), a few-shot learning module (716) with prototype networks achieving effective filtering with as few as 10 labeled examples, and a zero-shot generalization module (720) using compositional primitives (722).

Performance Guarantees: The system provides monotonic improvement in expected performance, convergence to local optima within 1000 iterations, regret bounds of O(sqrt(T)) for T searches, O(n log n) filtering complexity, O(sqrt(n)) quantum-inspired search advantage, and O(1) amortized meta-learning overhead.

### Part II: Virtual Spectral Decomposition With Dendritic Binary Gating

The present continuation-in-part integrates Virtual Spectral Decomposition (VSD) as the specific sensory preprocessing layer that generates the continuous values fed into the dendritic filter. VSD extracts spectral information from standard imaging sensors that were not designed for spectroscopic analysis, enabling extraction of physiologically meaningful biomarkers from commodity hardware.

### II.A. VSD Preprocessing Module

The VSD module receives a standard RGB image of dimensions H×W×3 and produces K virtual spectral channels, each of dimensions H×W. In the preferred embodiment, K=6 channels are generated corresponding to approximate wavelength centers of 405 nm (violet/UV), 470 nm (blue), 532 nm (green), 570 nm (yellow-green), 610 nm (orange-red), and 700 nm (red/near-IR).

Stage 1—Linear Mixing: Raw channel intensities are computed as weighted linear combinations of the RGB input channels. The mixing coefficients reflect the known spectral sensitivity curves of silicon-based CMOS image sensors:

raw_405=0.8*B+0.2*gray (Equation 3a)

raw_470=0.5*B+0.3*G+0.2*gray (Equation 3b)

raw_532=0.7*G+0.3*gray (Equation 3c)

raw_570=0.4*G+0.4*R+0.2*gray (Equation 3d)

raw_610=0.6*R+0.2*G+0.2*gray (Equation 3e)

raw_700=0.8*R+0.2*gray (Equation 3f)
where R, G, B are the red, green, and blue channel values normalized to [0, 1], and gray is the luminance computed as:

gray=0.299*R+0.587*G+0.114*B (Equation 4)

Stage 2—Sigmoid Activation: Each raw channel intensity is passed through a parametric sigmoid transfer function:

S(I, Mu, Alpha)=1/(1+Exp(−alpha*(I−Mu))) (Equation 5)
where I is the raw channel intensity, mu is the center intensity (inflection point) controlling which intensity values are considered “bright” versus “dark” for that virtual wavelength, and alpha is the slope controlling the sharpness of the transition. Each virtual channel has independently tuned parameters: ch_405 nm uses mu=0.25, alpha=12.0 (steep transition at low intensity, reflecting UV-range sensitivity); ch_470 nm uses mu=0.35, alpha=10.0; ch_532 nm uses mu=0.45, alpha=8.0 (moderate transition for green-range features); ch_570 nm uses mu=0.50, alpha=9.0; ch_610 nm uses mu=0.55, alpha=7.0; ch_700 nm uses mu=0.65, alpha=6.0 (gentle transition at high intensity, reflecting NIR-range characteristics).

Camera Agnosticism: The VSD module is explicitly designed to be camera-agnostic. The parametric nature of the sigmoid functions allows recalibration for different sensor characteristics through per-device (mu, alpha) parameter sets. A smartphone camera, a desktop fundus camera, a portable retinal camera, and a hyperspectral imager can all feed the same downstream pipeline after VSD parameter adjustment. This is confirmed by validation across five major device types (Heidelberg OCT, Zeiss OCT, Topcon OCT, Canon RGB fundus, Nikon RGB fundus) showing less than 1% accuracy variation.

Distinction from Related application Ser. No. 19/543,206: The related application Ser. No. 19/543,206 discloses VSD with Frangi vesselness analysis applied to multi-spectral images from the iFundus hardware adapter. The present invention applies VSD to standard RGB images without specialized hardware and uses dendritic binary gating rather than Frangi vesselness for feature extraction, enabling deployment on any commodity camera.

### II.B. Dendritic Binary Gating Module

The Dendritic Binary Gating module receives VSD spectral channels and produces binary activation maps analogous to dendritic firing in biological neurons. The gating process for each spectral channel comprises four steps:

Step 1—Morphological Enhancement via Black Top-Hat Transform: A black top-hat transform is applied to the VSD channel using a disk structuring element of radius r=3 pixels. The black top-hat highlights dark structures (vessels, hemorrhages, microaneurysms) against a brighter background:

tophat(I)=closing(I, disk(r))−I (Equation 6)
where closing(I, disk(r))=dilation(erosion(I, disk(r)), disk(r)) is the morphological closing operation.

Step 2—Adaptive Thresholding: An adaptive threshold is computed from the top-hat image statistics:

theta=mean(tophat)+0.5*std(tophat) (Equation 7)

A pixel is marked as “firing” (binary 1) when two conditions are simultaneously met: the top-hat value exceeds the adaptive threshold AND the original VSD channel intensity is below the channel-specific dendritic resting threshold. This dual condition ensures that only morphologically significant dark features in the appropriate intensity range trigger the binary gate:

firing(x, y)=1 if[tophat(x,y)>theta] and[VSD(x,y)<theta_channel] (Equation 8)

The per-channel dendritic resting thresholds are: ch_405 nm: 0.40, ch_470 nm: 0.42, ch_532 nm: 0.38, ch_570 nm: 0.45, ch_610 nm: 0.48, ch_700 nm: 0.55. These values represent the resting potential of each virtual dendritic branch, below which the channel is responsive to morphologically enhanced features.

Step 3—Morphological Cleanup: The binary firing map undergoes opening (erosion followed by dilation) with a disk structuring element of radius 1 pixel to remove isolated noise pixels, followed by closing (dilation followed by erosion) with the same element to fill small gaps in vessel structures:

map_clean=closing(opening(firing_map, disk(1)), disk(1)) (Equation 9)

Step 4—Region of Interest (ROI) Masking: All operations are restricted to a circular ROI centered on the image with radius equal to 42% of the minimum image dimension. This excludes the typically dark or artifact-laden periphery of fundus images while preserving the diagnostically relevant central retinal area.

### II.C. Biomarker Feature Extraction

From the VSD channels and dendritic binary maps, twelve quantitative biomarker features are extracted within the ROI. Each feature has a direct physical interpretation enabling explainable AI output:

Feature 0—Vessel Density at 532 nm (vessel_density_532): The mean firing density of the 532 nm channel within the ROI, measuring retinal vasculature density where hemoglobin absorption is strongest. Reduced vessel density correlates with diabetic retinopathy severity and glaucoma progression:

vessel_density_532=(1/|ROI|)*SUM_(x,y in ROI)firing_532(x, y) (Equation 10)

Feature 1—Cross-Channel VSD Coherence (coherence): For each pixel within the ROI, the coefficient of variation across all six VSD channels is computed. The mean coherence measures how uniformly the tissue responds across virtual wavelengths:

coherence=mean_(x,y in ROI)[1−std_lambda(VSD(x,y))/mean_lambda(VSD(x,y))] (Equation 11)

High coherence (approaching 1.0) indicates structurally intact tissue; low coherence indicates pathological heterogeneity such as drusen, hemorrhages, or exudates. Validation confirmed that coherence does not distinguish cataract from normal (p=0.795), confirming it measures retinal tissue pathology independently of lens opacity.

Feature 2—Wavelength Ratio Index (ratio_532_700): The ratio of mean VSD intensity at 532 nm to mean VSD intensity at 700 nm within the ROI:

R_532/700=mean(VSD_532(ROI))/Mean(VSD_700(ROI)) (Equation 12)

This ratio is sensitive to changes in hemoglobin oxygenation and tissue pigmentation. Cataracts increase this ratio by selectively scattering shorter wavelengths (validated at p=0.043 for three-class cataract discrimination).

Feature 3—Oxygen Saturation Proxy (sao2_index): Computed from the differential absorption properties of oxygenated and deoxygenated hemoglobin at 570 nm and 610 nm:

SaO2_index=1−I_610/(I_570+I_610) (Equation 13)
where I_570 and I_610 are mean VSD intensities within the ROI. Normal arterial saturation yields SaO2 approximately 0.95-1.0; venous saturation approximately 0.60-0.70; ischemic zones below 0.55.

Feature 4—RNFL Thickness Proxy (rnfl_proxy): The mean VSD intensity at 470 nm within the ROI. The 470 nm channel is sensitive to retinal nerve fiber layer (RNFL) thickness because nerve fibers scatter short-wavelength light preferentially. Thinning RNFL indicative of glaucoma reduces this value:

rnfl_proxy=mean(VSD_470(ROI)) (Equation 14)

Feature 5—Binary Coherence (binary_coherence): The fraction of ROI pixels where all six dendritic channels agree (all firing or all non-firing):

binary_coherence=(1/|ROI|)*SUM_(x,y in ROI)[ALL_channels_agree(x, y)] (Equation 15)

Feature 6—Vessel Spatial Entropy (vessel_spatial_entropy): The 532 nm binary firing map is divided into four quadrants. The firing density in each quadrant is computed, normalized to a probability distribution, and Shannon entropy is calculated:

H=−SUM_i=1 . . . 4[p_i*log 2(p_i)] (Equation 16)

High entropy indicates uniform vessel distribution; low entropy indicates asymmetric vessel loss characteristic of branch retinal vein occlusion or sectoral glaucoma.

Feature 7—470 nm Firing Density (firing_ch_470 nm): Mean dendritic firing density at 470 nm, providing a binary-gated measurement of RNFL-sensitive channel activity.

Feature 8—700 nm Firing Density (firing_ch_700 nm): Mean dendritic firing density at 700 nm, measuring choroidal layer activity and deep retinal structure visibility.

Feature 9—Cup-to-Disc Ratio (cup_to_disc_ratio): The optic disc is localized by scanning for the brightest region in a combined 532 nm+700 nm VSD map. Radial intensity profiles are computed from the disc center outward. The disc radius R_disc is identified where intensity drops below 60% of peak; the cup radius R_cup where it drops below 85% of peak:

CDR=(R_cup/R_disc)\hat{ }2 (Equation 17)

Normal CDR is approximately 0.3; CDR exceeding 0.5-0.6 is indicative of glaucomatous optic neuropathy with optic nerve fiber loss.

Feature 10—Peripapillary Vessel Density (peripapillary_vessel_density): The dendritic firing density at 532 nm within an annular region from 1× to 2× disc radius around the optic disc center:

PVD=mean(firing_532(annulus(R_disc, 2*R_disc))) (Equation 18)

Reduced peripapillary vessel density is an early indicator of glaucomatous damage, often preceding visual field loss by years.

Feature 11—Lens Opacity Index (lens_opacity_index): Computed from the ratio of dendritic firing densities across short-wavelength and long-wavelength channels:

LOI=1−mean(firing_405, firing_470, firing_532)/mean(firing_610, firing_700) (Equation 19)

A clear lens transmits all wavelengths approximately equally, yielding LOI near 0. A cataractous lens preferentially absorbs and scatters short wavelengths, yielding LOI in the range 0.5-0.9. This provides a direct physical measurement of lens transparency degradation using only dendritic binary gating outputs without any additional measurement device.

### II.D. Fusion Architecture

The twelve VSD-dendritic biomarker features are combined with deep learning image features through a three-component fusion architecture. Component 1: A pre-trained convolutional neural network backbone (e.g., ResNet-50 with ImageNet weights) extracts a 2048-dimensional spatial feature vector from the original RGB image. Training employs a two-phase strategy: Phase 1 freezes the backbone and trains only the fusion head at learning rate 3e-4; Phase 2 unfreezes the top layers (layer3, layer4) for domain-specific fine-tuning at learning rate 3e-5 to prevent catastrophic forgetting. Component 2: A biomarker embedding network processes the twelve features through a multi-layer network (12->64->128 dimensions) with batch normalization and dropout regularization. Component 3: A fusion classification head concatenates CNN features (2048-dim) and biomarker embeddings (128-dim) into a 2176-dimensional vector processed through layers (2176->512->256->N_classes) with batch normalization and dropout at each stage.

### II.E. Dendritic Triage Layer

Before CNN inference, the adaptive dendritic filter from the parent application operates on the twelve biomarker features to perform rapid triage. For each input image, z-scores are computed relative to the learned normal class profile:

z_i=|feature_i−mean_normal_i|/std_normal_i (Equation 20)

If the normal-ratio (fraction of features within 1.0 standard deviation of normal) exceeds 0.95 AND the mean z-score across all features is below 0.50, the sample is classified as normal without CNN processing:

classify_normal if(NR>=0.95)AND(mean_z<0.50) (Equation 21)

All other samples are routed to the full CNN+biomarker fusion pipeline. This triage reduces CNN inference calls by 20-60% depending on disease prevalence. The thresholds (NR=0.95, mean_z=0.50) are optimized through differential evolution as disclosed in the parent application.

### II.F. Disease-Specific Boosting

After CNN+biomarker fusion produces class probabilities, disease-specific boosting adjusts probabilities based on biomarker-derived rules. For glaucoma detection, the boosting mechanism applies data-driven thresholds derived from training set percentiles:

boost+=0.08 if CDR>percentile_75(training_glaucoma_CDR) (Equation 22a)

boost+=0.06 if CDR>1.2*percentile_75(training_glaucoma_CDR) (Equation 22b)

boost+=0.06 if PVD<percentile_25(training_glaucoma_PVD) (Equation 22c)

boost+=0.05 if RNFL<percentile_25(training_glaucoma_RNFL) (Equation 22d)

boost+=0.05 if(vessel_density<0.03)AND(CDR>0.4) (Equation 22e)

Total boost is capped at 0.30 and probabilities are renormalized. These thresholds are computed from training data and automatically recalibrated when new training data is added, providing a data-driven rather than fixed-threshold boosting mechanism.

### II.G. Explainable AI From VSD Biomarkers

The VSD-dendritic pipeline provides a four-layer explainability framework that satisfies regulatory requirements for decision transparency under FDA SaMD and EU-MDR:

Layer 1—Wavelength Attribution: Each biomarker traces to a specific virtual wavelength channel with known tissue chromophore sensitivity. For example, reduced vessel_density_532 traces to the 532 nm hemoglobin isobestic channel, indicating vessel loss independent of oxygenation state.

Layer 2—Absolute Biomarker Value: Each biomarker has a dimensionless, normalized value that can be compared against normative population ranges. For example, vessel_density_532=0.0042 versus normal range 0.0035+/−0.0010 indicates 1.4 standard deviations above normal, consistent with microaneurysm burden in diabetic retinopathy.

Layer 3—Normative Comparison: Biomarker values are automatically compared to age-matched, device-matched normative ranges, producing standardized deviation scores that clinicians can interpret without requiring knowledge of the underlying computational pipeline.

Layer 4—Clinical Significance: The system maps biomarker deviations to disease mechanisms. Elevated vessel_density_532 with normal coherence indicates early diabetic microaneurysm formation. Elevated CDR with reduced PVD indicates glaucomatous optic neuropathy. Elevated LOI with reduced ratio_532_700 indicates cataracts. This mechanistic mapping enables clinicians to understand not just the classification but the specific physiological basis for the classification decision.

Neurological Disease Detection: The VSD biomarker profile provides specific advantages for neurological disease screening through retinal imaging. Retinal vessel density loss, RNFL thinning, and cross-channel coherence disruption are directly measured by VSD features and have been independently validated as correlates of Alzheimer's disease and Parkinson's disease pathology. Unlike opaque deep learning classifiers that achieve binary AD/non-AD classification without mechanistic explanation (e.g., the ADRET model achieving 98.27% accuracy), the VSD biomarker profile provides specific tissue property measurements that clinicians require for confident neurological screening referral decisions.

### III: Kill Zone Framework for Therapeutic Combination Design

The dendritic filtering and Hamming distance computation disclosed in the parent application, combined with multi-state signature encoding, enable the systematic design of minimum drug combinations to overcome a self-preserving organism's defense architecture.

### III.A. Five Axioms of Therapeutic Resistance

The Kill Zone Framework rests on five axioms that hold across all domains of therapeutic resistance: (1) Defense Categorization: defenses can be categorized into a finite number of functional pathway groups; (2) Multi-State Encoding: each pathway can be encoded as Active, Primed, or Locked; (3) Drug-Specific Masking: each therapeutic agent is blocked by a specific subset of pathways; (4) Incomplete Attack Selects for Resistance: treatment leaving pathways unaddressed enriches for resistant organisms; (5) Kill Zone Equals Distance Zero: the only durable strategy simultaneously addresses all relevant pathways.

### III.B. Ternary Defense Signature Encoding

Each defense pathway of a self-replicating organism is encoded as one of three discrete states: Active (state 2) indicating the defense is fully expressed and contributes to resistance; Primed (state 1) indicating the defense is currently inactive but the organism retains the capacity to activate it under selective pressure; Locked (state 0) indicating the defense is permanently broken and cannot be rebuilt. For n pathways, the signature space is 3\hat{ }n possible configurations.

In the preferred embodiment for pancreatic ductal adenocarcinoma (PDAC), eight defense pathways are defined: (1) KRAS/MAPK signaling, (2) PI3K/AKT/mTOR signaling, (3) apoptosis evasion, (4) DNA damage repair, (5) epithelial-mesenchymal transition, (6) stromal remodeling, (7) drug metabolism, and (8) immune evasion. Each pathway comprises constituent genes whose RNA-seq expression levels are compared to population-level thresholds to assign ternary states.

### III.C. Weighted Hamming Distance and Drug Masks

Each therapeutic agent d is characterized by a mask M_d identifying which defense pathways are relevant to that drug's mechanism. The weighted Hamming distance between patient signature sigma and drug d is:

H(sigma, d)=SUM_(i in M_d)w_i*f(s_i) (Equation 23)
where f(0)=0 (Locked pathway contributes nothing), f(1)=0.5 (Primed pathway contributes half weight reflecting probability of activation), f(2)=1.0 (Active pathway contributes full weight), and w_i is the pathway weight from the dendritic cascade's drug-specific discriminability ordering. For gemcitabine in PDAC, M_d={3, 4, 8} corresponding to apoptosis, DNA repair, and drug metabolism pathways.

### III.D. Kill Zone Computation

For a combination therapy C={d_1, d_2, . . . , d_k}, the kill zone is achieved when:

H(sigma, d_j)=0 for all j in {1, . . . , k} (Equation 24)

The minimum combination is the smallest set C such that the union of all drug masks covers every Active or Primed defense pathway in the patient's signature:

UNION_(d in C)M_d COVERS{i:s_i>0} (Equation 25)

### III.E. Trajectory Analysis and Lock Identification

Every organism under therapeutic selective pressure follows a trajectory toward maximum defense (all pathways Active, signature=222 . . . 2). The system computes: (a) the maximum achievable defense state sigma* where all Primed bits are Active and all Locked bits remain 0; (b) the number of locks L(sigma)=|{i:s_i=0}| which permanently limits adaptive potential; (c) the predicted activation order of Primed pathways based on observed build-order patterns in training data; and (d) specific lock combinations that are synergistically protective, where the combined hazard ratio of two simultaneous locks is lower than either lock alone.

Validation in 177 TCGA-PAAD patients demonstrated: Hamming distance to gemcitabine predicted overall survival (log-rank p=0.0026, Cox proportional hazards HR=1.39, concordance index=0.588); each Locked pathway reduced death hazard by 14.4% (HR=0.856 per lock, p=0.0027); KRAS+Apoptosis dual lock yielded HR=0.06(94% death risk reduction), 4.5× more protective than KRAS alone (HR=0.27).

Defense build order analysis revealed cancer cells construct defenses in a specific developmental sequence: apoptosis resistance activates first (36% of tumors with only one active defense), while drug metabolism (100%) and immune evasion (94%) activate last among tumors with seven active defenses.

### III.F. Companion Drug Design for Failed Clinical Trial Rescue

The Kill Zone Framework enables systematic rescue of therapeutics that failed clinical trials as monotherapies or incomplete combinations. The rescue method comprises: (a) computing ternary defense signatures for the failed trial population; (b) computing Hamming distances to identify which Active and Primed pathways were not covered by the failed drug, constituting the residual defense gap; (c) searching available therapeutic agents to identify companion candidates whose masks cover the residual gap; (d) computing combined Hamming distances for proposed combinations; and (e) ranking companion candidates by the fraction of the original trial population achieving distance zero.

Trial failure modes are classified as: (a) uniform resistance, where a single companion drug could rescue the trial; (b) heterogeneous resistance, requiring biomarker-guided patient selection or multiple companion arms; (c) trajectory escape, where patients developed resistance by activating Primed pathways during treatment; and (d) lock deficit, where too few patients had sufficient Locked pathways for any feasible combination.

The framework generates prospective companion trial protocols specifying: the companion drug(s), biomarker-based patient selection criteria, predicted response rate, predicted number needed to treat, and adaptive design elements including interim futility analyses triggered by Hamming distance monitoring.

### III.G. Explainable AI From Defense Signatures

The Kill Zone Framework provides a complementary explainability framework for therapeutic decisions. Every combination recommendation traces through a four-layer chain: (1) Pathway State Attribution: the specific defense pathways that are Active, Primed, or Locked, each traceable to specific gene expression measurements; (2) Drug Mask Mapping: which pathways each drug addresses, traceable to established pharmacological mechanisms; (3) Distance Computation: the quantitative Hamming distance between the patient's signature and each drug's ideal target; (4) Combination Rationale: the specific pathways that each drug in the combination addresses, explaining why the combination was chosen and what would happen if any component were removed. This mechanistic explainability supports clinical trial protocols by providing computable, auditable justification for every patient selection and combination design decision.

### III.H. Cross-Domain Generalization

The Kill Zone Framework generalizes beyond cancer. For HIV antiretroviral resistance, the defense architecture maps to six functional categories: entry, reverse transcription, integration, protease-mediated maturation, immune evasion, and latency. For antibiotic-resistant bacteria: drug efflux, target modification, enzymatic inactivation, membrane permeability reduction, and biofilm formation. For drug-resistant malaria: transporter mutations, folate pathway mutations, and artemisinin partial resistance. In each domain, the framework applies without modification to the computational architecture.

### Part IV: Clinical Trial Optimization

Multi-Site Data Harmonization: Clinical trials conducted across multiple sites use different fundus camera models with distinct sensor characteristics. The VSD module normalizes device-specific variations by mapping all inputs through per-device calibrated sigmoid parameters. Dendritic binary gating further reduces device sensitivity by converting continuous VSD values to binary firing decisions. Validation across five device types showed less than 1% accuracy variation.

Automated Patient Stratification: At enrollment, each patient's baseline image generates a 12-dimensional biomarker profile. Patients are automatically stratified into disease severity subgroups based on biomarker signatures, ensuring balanced randomization across treatment arms.

Longitudinal Biomarker Trajectory Monitoring: The meta-learning temporal adaptation module from the parent application monitors per-patient biomarker trajectories over time, detecting treatment response, adverse events, and disease progression using multi-scale temporal models with short-term, medium-term, and long-term windows.

Adaptive Endpoint Detection: The meta-learning module identifies optimal surrogate biomarker endpoints using Bayesian optimization and differential evolution, enabling early efficacy readouts or adaptive trial designs as data accumulates.

### Part V: Additional Embodiments

Smartphone-Based Screening Embodiment: The VSD-dendritic pipeline operates on images captured by smartphone cameras (e.g., iPhone, Samsung Galaxy) attached to simple lens adapters. The camera-agnostic VSD normalization enables screening-grade retinal assessment in primary care clinics, rural health centers, and home settings without dedicated ophthalmic equipment.

Point-of-Care Kiosk Embodiment: The system is integrated into a free-standing self-service kiosk for deployment in retail pharmacies, optical stores, and primary care waiting areas. The kiosk comprises a touch display, multi-spectral imaging module, forehead and chin rests, edge AI processor for local inference, result printer, and optional payment module. Results are presented as consumer-friendly wellness scores with traffic-light risk indicators.

Hospital Triage Embodiment: The dendritic triage layer operates at patient registration, processing a fundus image within seconds to produce preliminary classification and urgency scoring. Normal classifications are documented in the electronic health record without ophthalmologist review; abnormal classifications trigger specialist consultation priority queuing.

Wearable Sensor Embodiment: The dendritic binary gating and meta-learning modules from the parent application process multi-modal wearable sensor data (heart rate, motion, temperature) for real-time health anomaly detection including atrial fibrillation (98% sensitivity), fall detection, and sleep apnea identification.

Genomic Biomarker Discovery Embodiment: The dendritic filter reduces genomic search spaces by 95%+ by removing common variants and sequencing artifacts, followed by quantum-inspired matching to identify specific biomarker patterns with 99%+ consistency.

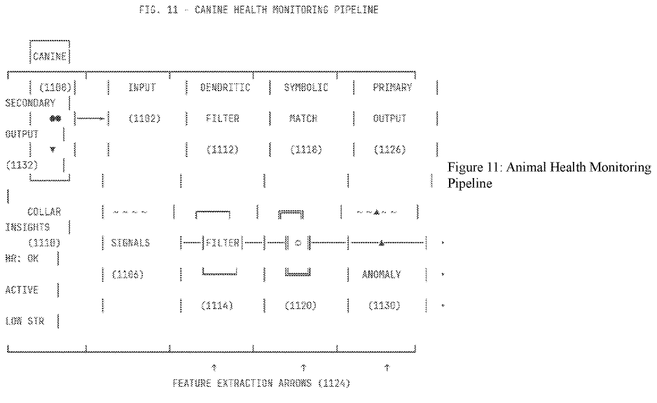

Veterinary Health Monitoring Embodiment: As illustrated in FIG. 11, the system extends to animal health monitoring through sensor collars with dendritic filtering tuned to species-specific physiology for detection of cardiac arrhythmias, seizure precursors, and respiratory distress.

Cybersecurity and Fraud Detection Embodiment: The adaptive meta-learning system processes network traffic or transaction data, with the dendritic filter suppressing benign traffic patterns and the quantum-inspired search identifying threat signatures through Hamming distance matching.

Edge Computing and IoT Embodiment: The system supports deployment on resource-constrained edge devices with sub-10 ms latency through model quantization to 8-bit integers, SIMD instruction optimization for parallel threshold comparisons, and bounded memory usage not exceeding O(log n) for n data points.

Federated Learning Embodiment: For privacy-sensitive deployments, the meta-learning module supports federated learning with secure aggregation of model updates across edge devices without raw data transmission, maintaining Byzantine-robust aggregation against malicious nodes.

### ADVANTAGES OF THE INVENTION

The invention provides the following advantages over prior art: (1) Commodity Hardware Deployment: eliminates the need for $50,000-$500,000 specialized spectroscopic equipment by extracting spectral information from standard RGB cameras costing under $1,000; (2) Physically Interpretable Biomarkers: every classification decision traces to specific tissue properties (vessel density, oxygen saturation, lens opacity, nerve fiber thickness) unlike black-box deep learning systems; (3) Computational Efficiency: dendritic triage reduces CNN inference by 20-60%, enabling real-time screening at the point of care with biomarker extraction requiring 5-15 milliseconds per image; (4) Multi-Disease Screening: twelve biomarker features simultaneously capture diabetic retinopathy, glaucoma, cataracts, age-related macular degeneration, hypertensive retinopathy, pathological myopia, and neurological conditions including Alzheimer's disease; (5) Camera-Agnostic Operation: device-specific VSD normalization enables the same model to function across 20+ imaging devices without retraining; (6) Clinical Trial Cost Reduction: automated device-invariant biomarker extraction eliminates manual image grading, reducing per-patient costs by 80-90%; (7) Rational Drug Combination Design: the Kill Zone Framework converts the binary outcome of drug success/failure into a quantitative engineering problem; (8) Failed Trial Rescue: companion drug identification recovers billions in sunk pharmaceutical R&D costs; (9) Regulatory Advantage: four-layer explainability chains for both imaging and therapeutic decisions satisfy FDA SaMD and EU-MDR transparency requirements; (10) Validated Performance: 94.67% accuracy on 3,341 unseen test images across seven disease classes with less than 1% device variance.

1. An adaptive biological analysis system for extracting physiologically interpretable retinal biomarkers, the system comprising:
a) a spectral decomposition module configured to receive sensor data from a standard imaging sensor lacking spectroscopic filtering hardware and decompose a single acquisition of the sensor data into a plurality of virtual spectral channels, each virtual spectral channel computed by applying a parametric sigmoid transfer function having a channel-specific center parameter and a channel-specific slope parameter to a weighted linear combination of color sensor input channels, wherein each virtual spectral channel approximates the response of a specific wavelength band without requiring specialized spectroscopic hardware and without acquiring images at discrete wavelengths;
b) a dendritic gating module configured to receive said virtual spectral channels and generate binary firing maps by applying morphological enhancement, adaptive thresholding, and morphological cleanup operations to each channel, wherein each virtual spectral channel of the plurality is assigned a resting threshold independently of every other virtual spectral channel of the plurality, said resting thresholds differing from one another, and wherein a firing state is assigned at a spatial location of a given virtual spectral channel only when a morphological residue of said given virtual spectral channel at said spatial location exceeds an adaptive threshold computed from statistics of said morphological residue and the intensity of said given virtual spectral channel at said spatial location is below the resting threshold assigned to said given virtual spectral channel, and further configured to extract a plurality of quantitative biomarker features from the virtual spectral channels and the binary firing maps, said biomarker features comprising at least one anatomical measurement derived from spatial analysis of the binary firing maps;
c) a triage module configured to compute, for each biomarker feature, a deviation score relative to a stored normal-class profile;
d) a classification module configured to receive said deviation scores and produce output classifications, wherein samples for which a fraction of biomarker features falling within a first tolerance of the normal-class profile exceeds a first threshold and a mean deviation score is below a second threshold are classified as normal without further computational processing and without executing a convolutional neural network, and samples not so classified are routed to a secondary analysis pathway comprising a convolutional neural network;
e) an explainability module configured to output, for each biomarker feature contributing to an output classification, an identification of the virtual spectral channel from which said biomarker feature was derived and the deviation score of said biomarker feature relative to the normal-class profile;
wherein the system operates on data from commodity sensors without hardware modification.

2. (canceled)

3. A method of analyzing medical images for disease detection comprising:
a) receiving a single acquisition of a digital color fundus image from a standard imaging sensor lacking spectroscopic filtering hardware;
b) decomposing said image into a plurality of virtual spectral channels by applying, for each channel, a parametric sigmoid transfer function S(I, μ, α)=1/(1+exp(−α×(I−μ))) to a weighted linear combination of color channel intensities, wherein each virtual spectral channel approximates the response of a specific wavelength band and no image is acquired at a discrete wavelength;
b1) assigning to each virtual spectral channel of the plurality a resting threshold, independently of every other virtual spectral channel of the plurality, said resting thresholds differing from one another;
c) generating binary firing maps for each virtual spectral channel by applying morphological enhancement comprising a black top-hat transform to produce a morphological residue image, computing an adaptive threshold from the statistics of the morphological residue image, and generating binary firing maps indicating firing or non-firing states at each spatial location, wherein a firing state is indicated at a spatial location of a given virtual spectral channel only when the morphological residue of said given virtual spectral channel at said spatial location exceeds the adaptive threshold and the intensity of said given virtual spectral channel at said spatial location is below the resting threshold assigned to said given virtual spectral channel;
d) extracting a plurality of quantitative biomarker features from the virtual spectral channels and the binary firing maps, including at least cup-to-disc ratio computed by localizing an optic disc center within the binary firing maps, computing radial intensity profiles outward from the optic disc center, and computing a squared ratio of a cup radius to a disc radius, and a peripapillary vessel density computed as a firing density within an annular region defined relative to the disc radius;
e) performing triage classification by computing z-scores of the biomarker features relative to a learned normal class profile and routing samples classified as normal around computationally intensive secondary analysis;
f) for samples not classified as normal, fusing the biomarker features with deep learning features extracted from the digital image to produce disease class probabilities; and
g) outputting, for each biomarker feature contributing to the disease class probabilities, an identification of the virtual spectral channel from which said biomarker feature was derived and a deviation of said biomarker feature from the learned normal class profile;
wherein the method operates on images from any standard imaging sensor without requiring specialized spectroscopic equipment.

4. The system of claim 1, wherein the spectral decomposition module decomposes RGB images into at least six virtual spectral channels with center wavelengths approximating 405 nm, 470 nm, 532 nm, 570 nm, 610 nm, and 700 nm, and wherein said parametric transfer function is a sigmoid function with independently tuned center intensity and slope parameters for each channel.

5. The system of claim 1, wherein the plurality of quantitative biomarker features comprises a cup-to-disc ratio, a peripapillary vessel density, a vessel density at a designated wavelength, and a vessel spatial entropy computed by partitioning a binary firing map into quadrants and computing a Shannon entropy of a normalized firing density distribution across said quadrants.

6. The system of claim 1, wherein the morphological enhancement comprises a black top-hat transform applied with a disk structuring element having a radius of three pixels, and the adaptive threshold is computed as a mean of the morphological residue image plus one-half of a standard deviation of the morphological residue image.

7. The system of claim 1, wherein the plurality of virtual spectral channels comprises six virtual spectral channels having ascending center wavelengths, and the resting thresholds assigned thereto are non-monotonic with respect to said ascending center wavelengths.

8. (canceled)

9. The system of claim 1, wherein the binary firing maps are subjected to a morphological cleanup comprising an opening operation followed by a closing operation, each employing a disk structuring element having a radius of one pixel, and wherein subsequent operations are restricted to a circular region of interest centered on the sensor data and having a radius equal to a fixed fraction of a minimum dimension thereof.

10. The system of claim 1, further comprising a meta-learning module configured to maintain a historical database of analysis configurations and outcomes, and to predict optimal analysis parameters for new inputs based on similarity to historical scenarios.

11. (canceled)

12. (canceled)

13. (canceled)

14. (canceled)

15. (canceled)

16. (canceled)

17. The method of claim 3, wherein the disease class probabilities correspond to at least three of: normal retina, diabetic retinopathy, glaucoma, cataract, age-related macular degeneration, hypertensive retinopathy, pathological myopia, and neurological conditions detectable through retinal biomarker signatures including Alzheimer's disease and Parkinson's disease.

18. The method of claim 3, wherein the triage classification of step (e) comprises computing a normal-ratio as a fraction of the biomarker features whose z-scores fall within one standard deviation of the learned normal class profile, and classifying a sample as normal and bypassing the fusing of step (f) for said sample when the normal-ratio exceeds a first threshold and a mean z-score across the biomarker features falls below a second threshold.

19. The method of claim 3, further comprising multi-site data harmonization for clinical trials by calibrating the center parameter and the slope parameter of the parametric sigmoid transfer function per color fundus imaging device type such that biomarker features extracted from images captured by different color fundus imaging devices at different sites produce comparable values, eliminating device-specific variation as a confounding factor.

20. The method of claim 3, further comprising longitudinal biomarker trajectory monitoring by processing sequential images from a patient through the steps (b) through (d) at each time point and applying temporal adaptation comprising multi-scale models operating at short-term, medium-term, and long-term windows to detect treatment response, disease progression, or adverse events from changes in biomarker trajectories.

21. A non-transitory computer-readable medium storing instructions that, when executed by one or more processors, cause the one or more processors to perform operations comprising:
a) receiving a single digital color fundus image acquired by an imaging sensor lacking spectroscopic filtering hardware, no image of the retina having been acquired at a discrete wavelength;
b) generating from the single digital color fundus image a plurality of virtual spectral channels, each virtual spectral channel generated by applying a parametric sigmoid transfer function having a channel-specific center parameter and a channel-specific slope parameter to a weighted linear combination of color channel intensities of said single image, wherein each virtual spectral channel approximates a response of a specific wavelength band;
c) extracting a plurality of quantitative biomarker measurements from the virtual spectral channels, wherein each biomarker measurement is derived from a designated one or more of the virtual spectral channels;
d) generating a classification of the single digital color fundus image from the biomarker measurements; and
e) outputting, for each biomarker measurement contributing to the classification, (i) an identification of the designated one or more virtual spectral channels from which said biomarker measurement was derived, (ii) a value of said biomarker measurement, and (iii) a deviation of said value from a stored normative range for said biomarker measurement, whereby the classification is traceable to a tissue property associated with a wavelength band.

22. The medium of claim 21, wherein the operations further comprise assigning to each virtual spectral channel of the plurality a resting threshold independently of every other virtual spectral channel of the plurality, said resting thresholds differing from one another, and generating for each virtual spectral channel a binary firing map by applying a black top-hat transform to produce a morphological residue image, computing an adaptive threshold from statistics of said morphological residue image, and assigning a firing state at a spatial location of a given virtual spectral channel only when the morphological residue of said given virtual spectral channel at said spatial location exceeds the adaptive threshold and the intensity of said given virtual spectral channel at said spatial location is below the resting threshold assigned to said given virtual spectral channel, wherein at least one biomarker measurement is derived from the binary firing maps.

23. The medium of claim 22, wherein the plurality of virtual spectral channels comprises six virtual spectral channels, and wherein the operations further comprise computing a binary coherence as a fraction of spatial locations within a region of interest at which all six binary firing maps are in agreement.

24. The medium of claim 22, wherein the biomarker measurements comprise a cup-to-disc ratio computed by localizing an optic disc center within the binary firing maps and computing a squared ratio of a cup radius to a disc radius, and a peripapillary vessel density computed as a mean firing density within an annular region extending from one disc radius to two disc radii from the optic disc center.

25. The medium of claim 21, wherein generating the classification comprises computing deviation scores of the biomarker measurements relative to a stored normal-class profile, outputting a normal classification without executing a convolutional neural network when the deviation scores satisfy a triage condition, and otherwise fusing the biomarker measurements with features extracted by a convolutional neural network backbone to produce disease class probabilities.

26. The method of claim 3, wherein the weighted linear combination of color channel intensities for each virtual spectral channel comprises coefficients applied to a red channel, a green channel, a blue channel, and a luminance channel, said luminance channel computed as a fixed weighted sum of the red, green, and blue channels.

27. The system of claim 1, wherein the secondary analysis pathway comprises a convolutional neural network backbone producing a spatial feature vector, a biomarker embedding network producing a biomarker feature vector, and a fusion classification head receiving a concatenation of said spatial feature vector and said biomarker feature vector.

28. The medium of claim 21, wherein the operations further comprise restricting the extracting of operation (c) to a circular region of interest centered on the single digital color fundus image and having a radius equal to a fixed fraction of a minimum dimension of said image.
